(19) United States
(12) Patent Application Publication
(10) Pub. No.: US 2006/0081247 A1
(43) Pub. Date: Apr. 20, 2006

(54) Humidifier for breathing apparatus and method of humidifying a breathing apparatus gas strem

(76) Inventor: Danny Britt, Jackson, TN (US)

Correspondence Address: PETER L. BREWER BEARMAN, CALDWELL, & BERKOWITZ;LAW FIRM, 165 MADISON AVENUE, SUITE 2000, MEMPHIS, TN, 38103, US

(21) Appl. No.: 10/969,418

(22) Filed: Oct. 20, 2004

(51) Int. Cl. A61M 16/10; A61M 15/00; H05B 3/00

(52) U.S. Cl. 128/203.16; 128/203.17

(57) ABSTRACT

A humidifier (10) is disclosed which is adapted to be used in conjunction with a patient gas delivery system. The humidifier includes a housing (15) and two masses of hydrophilic polymer material (16) and (17) within the housing (15). The housing is configured to be isolated from an external liquid source during operation of the humidifier. The housing (15) also includes an inlet end (19) and an outlet end (20) through which a gas stream is pumped. As the gas stream passes through the saturated hydrophilic material, the gas stream becomes saturated with the fluid, thereby humidifying the gas stream.

### TECHNICAL FIELD

This invention relates generally to humidifiers, and more particularly to a humidifier for use with a breathing apparatus and methods of humidifying a gas stream for a breathing apparatus.

### BACKGROUND OF THE INVENTION

Oftentimes a patient is placed on a breathing apparatus to ensure that the patient receives a proper amount of air. The gas supplied by such devices must be humidified to ensure that it does not dry out the air passages of the patient.

In the past, humidification has been achieved through a ventilating bubbler-type gas humidifier. These humidifiers typically include a container holding a supply of water or other fluid through which the gas supply is passed to increase the moisture content, as shown in U.S. Pat. No. 3,929,128. This type of humidifier however requires a constant supply of water, which may be cumbersome, requires substantial cleaning, and is apt to producing a hazardous condition should the water be accidentally released.

Humidifiers may also utilize a wicking material immersed within the water supply to increase the surface area in direct contact with the gas stream and thereby increase the moisture content within the gas stream, as shown in U.S. Pat. No. 4,381,267. Again, this type of humidifier requires a constant supply of water and therefore includes the same problems associated therewith.

Humidifiers may also use a wicking material without a pool of water. For example, humidifiers have been designed to include a saturated sponge over which the gas stream passes. It has been found however that sponges dry out very quickly, thus requiring often re-wetting of the sponge throughout the day.

Humidifiers have also been designed which include a housing encasing one or more tubes made or a material which is permeable to water vapor but impermeable to water, as shown in U.S. Pat. No. 4,155,961. Gas is passed through the container which entrains the water vapor which advances through the walls of the tubes. Again, this type of humidifier requires a constant supply of water and therefore includes the same problems associated therewith.

Accordingly, it is seen that a need remains for a humidifier which does not include a constant supply of water. It is to the provision of such therefore that the present invention is primarily directed.

### SUMMARY OF THE INVENTION

In a preferred form of the invention a humidifier for use with a patient gas delivery system comprises a mass of hydrophilic polymer material, and a housing defining an interior chamber containing the mass of hydrophilic polymer material. The housing has a gas inlet in fluid communication with the interior chamber and a gas outlet in fluid communication with the interior chamber. With this construction, the mass of hydrophilic polymer material is saturated with a fluid, and a gas stream is passed into the housing interior chamber through the gas inlet and into direct contact with the fluid saturated mass of hydrophilic polymer material, wherein fluid vapors emanating from the hydrophilic polymer material are entrained into the gas stream which is then expelled from the chamber through the gas outlet.

In another preferred form of the invention, a method of humidifying a patient's gas supply comprises the steps of providing a housing defining a chamber, positioning a mass of hydrophilic polymer material within the housing, placing the housing within a pool of fluid to cause saturation of the hydrophilic polymer material, draining fluid from the housing, coupling the housing to a length of gas delivery tubing, and passing a gas stream through the housing and through the mass of saturated hydrophilic polymer material to cause entrainment of the fluid vapor emanating from the hydrophilic polymer material into the gas stream.

### BRIEF DESCRIPTION OF THE DRAWING

FIG. 1 is a perspective view of a humidifier embodying principles of the invention in a preferred form.

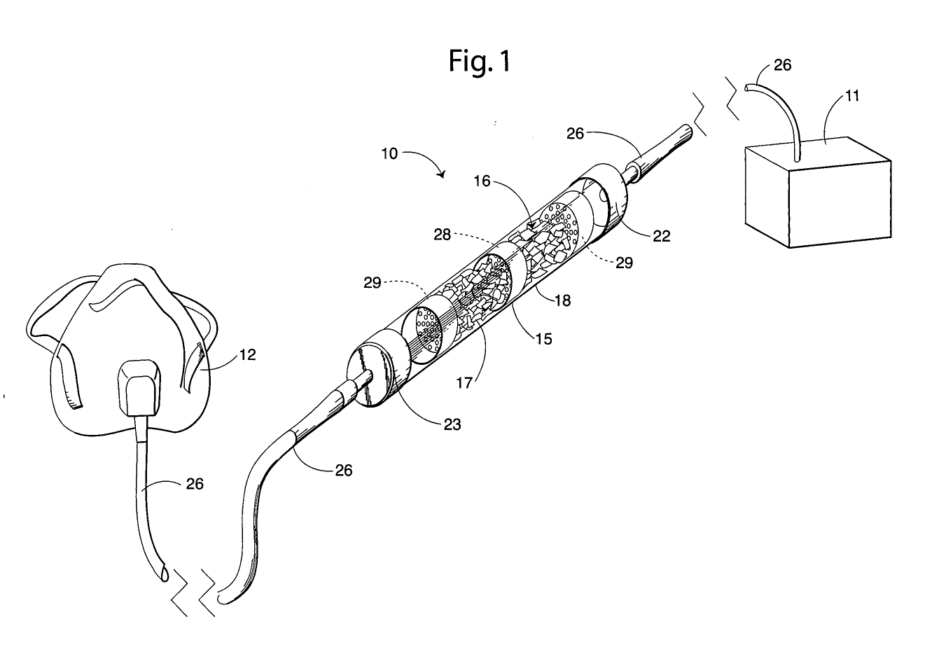

FIG. 2 is an exploded, perspective view, in partial cross-section, of the humidifier of FIG. 1.

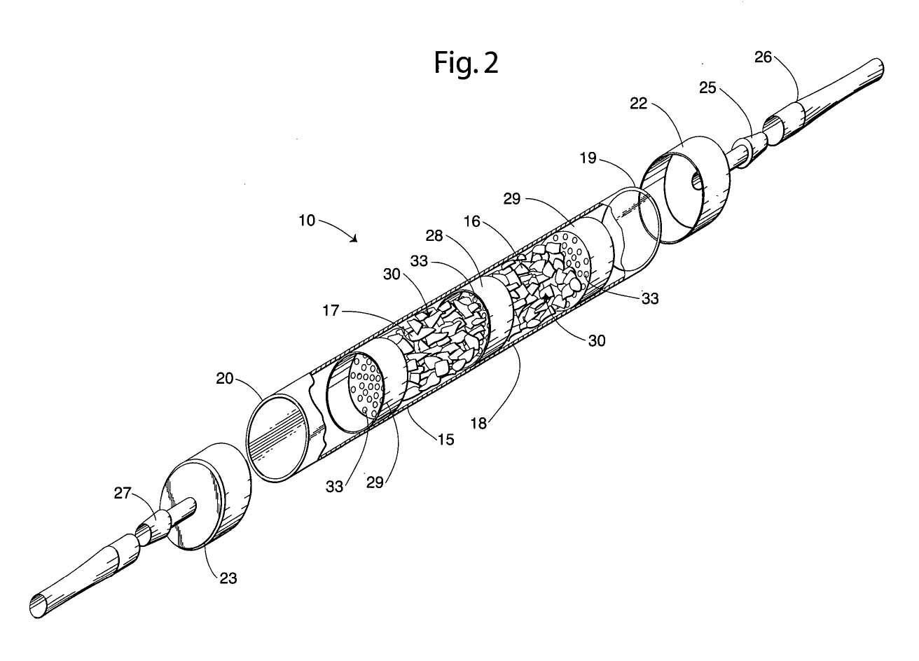

### DETAILED DESCRIPTION

With reference next to the drawings, there is shown a humidifier 10 in a preferred form of the invention. The humidifier 10 is adapted to be used in conjunction with a patient gas delivery system that may include a conventional gas stream generator 11, such as a concentrator, and a conventional patient delivery apparatus 12, such as a nasal cannula or mask.

The humidifier 10 includes a housing 15 and two masses of hydrophilic polymer material 16 and 17, shown in the form of randomly shaped grains. The term ‘grain’ is intended to convey both randomly shaped, lose pieces of material and uniformly shaped, lose pieces of material. The hydrophilic polymer material may be made of polyether polyurethane such as Hydromed D-J or Hydromed D640 brand materials commercially available from CT Biomaterials, a Division of CardioTech International, Inc., 229 Andover Street Wilmington, Mass. 01887, USA. The housing 15 includes a tubular body 18 having an inlet end 19 and an outlet end 20. An inlet cap 22 is removably mounted to the body inlet end 19, while an outlet cap 23 is removably mounted to the body outlet end 20. The inlet cap 22 has a hollow tube coupler 25 sized and shaped to mate with the end of a length of conventional respirator tubing 26 extending from the gas stream generator 11. The outlet cap 23 similarly has a hollow tube coupler 27 sized and shaped to mate with the end of a length of conventional respirator tubing 26 which extends to the patient delivery apparatus 12.

The humidifier 10 also includes a separator 28 and two end stops 29, which are all mounted within the tubular body 18. The combination of the tubular body 18, two end stops 29, and separator 28 creates two chambers 30 which hold and separate the first mass of hydrophilic polymer 16 from the second mass of hydrophilic polymer 17. The two end stops 29 prevent the two masses of hydrophilic material from accidentally escaping the tubular body 18 through the open ends 19 and 20. The separator 28 and stops 29 each have a series of small openings 33 therethrough which are configured to allow the passage of gas and water therethrough but which prevent the passage of the hydrophilic polymer, i.e., the hydrophilic polymer masses are contained within two chambers 30.

In use, the inlet cap 22 and outlet cap 23 are removed from the tubular body 18. The tubular body 18 is then immersed in a bath of water or other fluid for a time period of approximately five minutes, referred hereinafter simply as water for ease of explanation. The water enters the open ends 19 and 20, flows through the openings 33 within the end stops 29 and into chambers 30. A portion of the water within the chambers 30 is absorbed by the first and second masses of hydrophilic polymer 16 and 17. The tubular body 18 is then removed from the water bath and tilted to cause water within the chambers 30 to be expelled from the tube. The inlet cap 22 and outlet cap 23 are then repositioned upon their respective ends 19 and 20 in order to seal the housing except for the passages through end cap couplers 25 and 27.

The humidifier inlet cap coupler 25 is joined to the end of the respirator tubing 26 extending from the gas stream generator 11. Similarly, the outlet cap coupler 27 is joined to the end of the respirator tubing 26 extending to the delivery apparatus 12.

As gas from the gas stream generator 11 passes through the respirator tubing 26 it enters the humidifier 10 through the inlet cap 22. The gas stream passes through the opening 33 in the end stop 29 and into the chamber 30 wherein is passes through the first mass of hydrophilic polymer 16. The gas stream then continues through the openings 33 within the separator 28 and into the second chamber wherein is passes through the second mass of hydrophilic polymer 17. Lastly, the gas stream passes through the openings 33 within the end stop 9 and through the outlet cap 23 into the respirator tubing 26. The gas stream passes through the respirator tubing 26 to the delivery apparatus 12.

It should be understood that as the gas stream passes through the first and second masses of hydrophilic polymer 16 and 17 it entrains water vapor expelled from these masses. As such, the gas stream becomes humidified with water vapor from the hydrophilic polymer as it passes through the humidifier. However, this humidification does not require a constant supply or pool of water to be present, a problem long associated with humidifier of the prior art. Also, evaporation of the water from the hydrophilic material is generally constant and therefor the material does not dry out quickly with the initiation of the gas stream. As such, the material does not have to be re-wetted throughout the day as with humidifiers that utilize sponges and the like.

A test was conducted with a humidifier of the present invention having the following parameters: a tubular body length of 21.5 cm, an effective cylinder volume of 96 cc, and 8 grams of hydrophilic polymer material in each of two chambers. To simulate normal patient conditions, the humidifier was coupled to fifty feet of respirator tubing coupled to a water trap and a seven foot cannula. The housing 15 was immersed in a water bath and hydrophilic polymer was calculated to have absorbed 59 grams of water.

The humidifier was coupled to a Devilbliss MC44DS-90 concentrator at room temperature (72 degrees Fahrenheit during the day and 66 degrees Fahrenheit during the night). The concentrator output was set to two liters per minute and operation was observed for a 24 hour period.

Tests results under these conditions produced a total output of 29 milliliters during the 24 hour period, with 2 milliliters of ‘rainout’ water being drained from the respirator tubing, water trap and cannula. The term ‘rainout’ is intended to refer to water that condenses or otherwise collects within the tubing, water trap, and cannula, and is therefore not included in the gas stream humidity. This output is comparable to conventional bubbler diffusion humidifiers such as that produced by Salter Labs of Arvin, Calif. However, the present invention only produced 2 milliliters of rainout water while the bubbler diffusion humidifier produced 10 milliliters of rainout water. As such, it is possible that the present invention may be capable of being operated without the need of a water trap, which would effectively reduce the overall cost, reduce connection problems, and eliminate a cleanliness problem associated with water traps. The production of rainout water can also be reduced with the present invention by placing the humidifier closer to the patient.

It should be understood that the present invention may include any number of chambers within the tubular housing or may include any number of humidifiers placed in series in order to achieve a desired humidification level. It is believed that the creation of the chambers prevents the formation of a large single mass of hydrophilic polymer that may cause a blockage of the gas stream through the housing. The granular size of the hydrophilic polymer must also take into account the swelling of the material as it absorbs fluids, therefore the chamber should not contain so much material as to prevent gasflow once the material has absorbed the fluid. Also, the housing is not required to be tubular in shape, as it could be a housing of any shape or configuration having an gas inlet and an outlet and able to contain a mass of hydrophilic polymer therein.

It should be understood that even though the hydrophilic polymer is shown as a mass of granular pieces such is not intended to be a limitation, as the mass of hydrophilic polymer may be formed as sheets and folded within the tubular body or may be a form-fitted insert that conforms to the interior of the tubular body.

It should be understood that the term ‘gas’ is intended to include air or other gaseous materials such as nitrogen, oxygen, or medicates.

It thus is seen that a humidifier is now provided which overcomes problems with humidifier of the prior art by humidifying an gas stream without the need of a liquid pool. While this invention has been described in detail with particular references to the preferred embodiments thereof, it should be understood that many modifications, additions and deletions, in addition to those expressly recited, may be made thereto without departure from the spirit and scope of the invention as set forth in the following claims.

1. A humidifier for use with a patient gas delivery system, the humidifier comprising:
a mass of hydrophilic polymer material; and
a housing defining an interior chamber containing said mass of hydrophilic polymer material, said housing having a gas inlet in fluid communication with said interior chamber and a gas outlet in fluid communication with said interior chamber, and said housing being continuously isolated from an external liquid source during operation of the humidifier;
whereby the mass of hydrophilic polymer material is saturated with a fluid, and whereby a gas stream is passed into the housing interior chamber through the gas inlet and into direct contact with the fluid saturated mass of hydrophilic polymer material, wherein fluid vapors emanating from the hydrophilic polymer material are entrained into the gas stream and then expelled from the chamber through the gas outlet during operation.

2. The humidifier of claim 1 wherein said mass of hydrophilic polymer material is a mass of granular hydrophilic polymer material.

3. The humidifier of claim 1 wherein said housing is tubular and wherein said gas inlet is adjacent one end of said tubular housing and said gas outlet is adjacent an opposite end of said tubular housing.

4. The humidifier of claim 3 wherein said housing includes a first stop adjacent said gas inlet and a second stop adjacent said gas outlet, and wherein said stops have openings therethrough configured to allow the passage of fluid therethrough but prevents the passage of the hydrophilic polymer material therethrough.

5. The humidifier of claim 4 wherein said housing further comprises a separator for separating the mass of hydrophilic polymer material into a first portion and a second portion.

6. The humidifier of claim 5 wherein said separator has openings therethrough configured to allow the passage of fluid therethrough but prevents the passage of the hydrophilic polymer material therethrough.

7. The humidifier of claim 1 wherein said housing defines a plurality of interior chambers in fluid communication with each other.

8. A humidifier for use with a patient gas delivery system, the humidifier comprising:
a tubular housing having a gas inlet opening and a gas outlet opening, the housing being continuously isolated from an external liquid source during operation of the humidifier; and
a mass of hydrophilic polymer material positioned within said housing, said mass of hydrophilic polymer material being a granular mass of hydrophilic material,
whereby the mass of hydrophilic polymer material is saturated with a fluid, and whereby a gas stream is passed into the housing interior chamber through the gas inlet and into direct contact with the fluid saturated mass of hydrophilic polymer material, wherein fluid vapors emanating from the hydrophilic polymer material are entrained into the gas stream which is then expelled from the chamber through the gas outlet during operation.

9. The humidifier of claim 8 wherein said tubular housing is cylindrical and wherein said gas inlet is adjacent one end of said cylindrical housing and said gas outlet is adjacent an opposite end of said cylindrical housing.

10. The humidifier of claim 9 wherein said housing includes a first stop adjacent said gas inlet and a second stop adjacent said gas outlet, and wherein said stops have openings therethrough configured to allow the passage of fluid therethrough but prevents the passage of the hydrophilic polymer material therethrough.

11. The humidifier of claim 10 wherein said housing further comprises a separator for separating the mass of hydrophilic polymer material into a first portion and a second portion.

12. The humidifier of claim 11 wherein said separator has openings therethrough configured to allow the passage of fluid therethrough but prevents the passage of the hydrophilic polymer material therethrough.

13. The humidifier of claim 8 wherein said housing defines a plurality of interior chambers in fluid communication with each other.

14. A method of humidifying a patient's gas supply comprising the steps of:
(a) providing a housing defining a chamber;
(b) positioning a mass of hydrophilic polymer material within the housing;
(c) placing the housing within a pool of fluid to cause saturation of the hydrophilic polymer material;
(d) draining fluid from the housing;
(e) coupling the housing to a length of gas delivery tubing; and
(f) passing a gas stream through the housing and through the mass of saturated hydrophilic polymer material to cause entrainment of the fluid vapor emanating from the hydrophilic polymer material into the gas stream.

15. The method of claim 14 wherein step (b) the mass of hydrophilic polymer material is a mass of granular hydrophilic polymer material.
